(19) United States
(12) Patent Application Publication
(10) Pub. No.: US 2021/0154127 A1
(43) Pub. Date: May 27, 2021

(54) ALCOHOL-BASED SPRAYABLE GEL WITH YIELD POINT

(71) Applicant: Henkel AG & Co. KGaA, Duesseldorf (DE)

(72) Inventors: KONSTANTIN GOUTSIS, Juechen (DE); DANIELA KESSLER-BECKER, Leverkusen (DE)

(73) Assignee: Henkel AG & Co. KGaA, Duesseldorf (DE)

(21) Appl. No.: 16/952,370

(22) Filed: Nov. 19, 2020

(30) Foreign Application Priority Data
Nov. 22, 2019 (DE) 10 2019 218 009.4

(51) Int. Cl. A61K 8/81; A61Q 1/02; A61Q 5/00; A61K 8/34; A61K 8/41; A61K 8/87; A61K 8/25; A61K 8/29; A61K 8/04; A61K 8/92

(52) U.S. Cl. CPC A61K 8/8152; A61Q 1/02; A61Q 5/00; A61K 8/34; A61K 8/41; A61K 8/87; A61K 2800/436; A61K 8/29; A61K 8/042; A61K 8/92; A61K 2800/30; A61K 2800/412; A61K 8/25

(57) ABSTRACT

A cosmetic composition is provided for application to the hair or skin, comprising 30 to 85% ethanol, 15 to 37% water, a crosslinked copolymer composed of acrylic acid and non-ethoxylated esters of acrylic acid with linear C10-C30 monoalcohols as monomers, the crosslinked copolymer being present in a total amount of 0.1 to 1.0%, a alkalizing agent up to a pH of the cosmetic composition in the range of 5 to 8, measured at 20° C., a substance selected from oils and fragrances in a total amount of 0 to 10%, one or more surfactants in a total amount of 0 to 0.1%, and at least one pigment in a total amount of 0.1 to 10%, where all quantities are based on the weight of the cosmetic composition, whereby the application is preferably done by spraying, especially preferably by spraying with a pump sprayer.

### CROSS-REFERENCE TO RELATED APPLICATION

This application claims priority to German Patent Application No. 10 2019 218 009.4, filed Nov. 22, 2019, which is incorporated herein by reference in its entirety.

### TECHNICAL FIELD

The present application concerns a cosmetic composition for the treatment of the hair or the skin which allows the application of pigments, in particular pearlescent pigments, which give the treated hair or skin a temporary color or lustre effect.

### BACKGROUND

To make the application to the hair as easy as possible for the user, the cosmetic composition as contemplated herein should be sprayable. In addition, the cosmetic carrier medium for the pigments should leave as little residue as possible on the treated hair or skin to minimize the color effect of the pigments.

The pigments contained in the cosmetic composition, especially if they are mineral pigments, have a density in the range of approximately 2.5 to about 3.5 grams/cm³, which is about three times the density of the carrier medium. The rheological properties of the cosmetic composition as contemplated herein should on the one hand enable a homogeneous dispersion of the pigments in the composition which is as storage-stable as possible, so that the cosmetic composition should have the viscosity of a gel on the one hand and a yield point on the other hand and be sufficiently shear-sensitive so that it can be applied as a pump spray.

Furthermore, the pigments contained should adhere well to the treated surface (skin/hair) so that they do not trickle off after the carrier medium has dried.

Finally, the cosmetic composition as contemplated herein should represent a visually and olfactorically attractive cosmetic. The carrier medium should be as transparent as possible and have a pleasant perfume.

### BRIEF SUMMARY

Surprisingly, these tasks were solved by a cosmetic composition containing
about 30-85% by weight ethanol,
about 15-37% by weight water,
at least one crosslinked copolymer composed of acrylic acid and non-ethoxylated esters of acrylic acid with linear C10-C30 monoalcohols as monomers, the crosslinked copolymer being present in a total amount of about 0.1-1.0% by weight, preferably about 0.3-0.8% by weight, particularly preferably about 0.45-0.60% by weight,
at least one alkalizing agent up to a pH of the cosmetic composition in the range about 5-8, preferably in the range about 6-7, particularly preferably in the range about 6.2-6.8, each measured at about 20° C.,
at least one substance selected from oils and fragrances in a total amount of about 0-10% by weight,
one or more surfactants in a total amount of about 0-0.1% by weight, and
at least one pigment in a total amount of about 0.1-10% by weight,
   where all quantities are based on the weight of the cosmetic composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

### Ethanol

In order that the color effect of the pigments on the treated hair or skin is impaired as little as possible, the cosmetic carrier medium for the pigments as contemplated herein should leave as little residue as possible on the treated hair or skin. Therefore, the cosmetic compositions as contemplated herein contain about 30-85% by weight, preferably about 40-80% by weight, particularly preferably about 55-78% by weight, extremely preferably about 60-77% by weight ethanol, each based on the weight of the cosmetic composition.

### Water

To ensure a storage-stable homogeneous dispersion of the pigments in the cosmetic carrier medium as contemplated herein, a thickening agent must be included. Preferred thickening agents, which also enable the transparent design of the cosmetic carrier medium, require a certain amount of water in the medium to be thickened. Therefore the cosmetic compositions as contemplated herein contain about 15-37% by weight, preferably about 18-30% by weight, particularly preferably about 20-28% by weight, extremely preferably about 22-25% by weight of water, each based on the weight of the cosmetic composition.

Crosslinked copolymer of acrylic acid and non-ethoxylated esters of acrylic acid with linear C10-C30 monoalcohols

Surprisingly, it was found that the tasks presented here are optimally solved using at least one cross-linked copolymer composed of acrylic acid and non-ethoxylated esters of acrylic acid with linear C10-C30 monoalcohols as monomers.

A further essential feature of the cosmetic composition as contemplated herein is the content of at least one crosslinked copolymer composed of acrylic acid and non-ethoxylated esters of acrylic acid with linear C10-C30 monoalcohols as monomers, the at least one crosslinked copolymer being present in a total amount of about 0.1-1.0% by weight, preferably about 0.3-0.8% by weight, particularly preferably about 0.45-0.60% by weight, in each case based on the weight of the cosmetic composition. At least one crosslinked copolymer of acrylic acid and non-ethoxylated esters of acrylic acid with linear C10-C30 monoalcohols is preferably selected from copolymers with the INCI name Acrylates/C10-30 Alkyl Acrylate Crosspolymer. Preferred cross-linking agents are selected from sucrose allyl ether and pentaerythrityl allyl ether.

Cross-linked copolymers of acrylic acid and non-ethoxylated esters of acrylic acid with linear C10-C30 monoalcohols, which are particularly preferred as contemplated herein, are obtainable by polymerization of a monomer mixture which contains—in each case based on its weight—about 80 to 99 wt. % of a monomer mixture which is %, preferably about 90 to about 98% by weight, of acrylic acid, at least one non-ethoxylated ester of acrylic acid with linear C10-C30 monoalcohols in a total amount of about 0.9-19.9% by weight, preferably about 2-10% by weight, and at least one crosslinking agent in a total amount of about 0.1-4% by weight. Preferred cross-linking agents are selected from sucrose allyl ether and pentaerythrityl allyl ether.

Other crosslinked copolymers of acrylic acid and non-ethoxylated esters of acrylic acid with linear C10-C30 monoalcohols which are particularly preferred as contemplated herein about 0.5% by weight dispersion in water at about 25° C. and a pH in the range about 5.8-6.3 has a viscosity in the range about 45,000 to about 65,000 mPas, measured with a Brookfield RVF or Brookfield RVT viscometer at a rotation frequency of about 20 min⁻¹ with spindle #7.

The content of the at least one cross-linked copolymer, built up from acrylic acid and non-ethoxylated esters of acrylic acid with linear C10-C30 monoalcohols as monomers, is selected such that the viscosity of the cosmetic composition as contemplated herein is preferably in the range of about 3.000-6,000 mPas, preferably about 3,500-5,500 mPas, particularly preferably about 4,000-4,500 mPas, in each case measured at about 20° C. with the Brookfield DV-II Pro rotational viscometer, spindle 4 at a rotational frequency of 4 min⁻¹.

The cosmetic composition as contemplated herein contains at least one crosslinked copolymer, built up from acrylic acid and non-ethoxylated esters of acrylic acid with linear C10-C30 monoalcohols as monomers, in a total amount of about 0.1-1.0% by weight, preferably about 0.3-0.8% by weight, particularly preferably about 0.45-0.60% by weight, in each case based on the weight of the cosmetic composition.

Cosmetic composition, preferred as contemplated herein, containing in a total amount of about 0.1-1.0% by weight, preferably about 0.3-0.8% by weight, particularly preferably about 0.45-0.60% by weight %, in each case based on the weight of the cosmetic composition, of at least one crosslinked copolymer composed of acrylic acid and non-ethoxylated esters of acrylic acid with linear C10-C30 monoalcohols as monomers, this copolymer being obtainable by polymerization of a monomer mixture which contains—in each case based on its weight—from about 80 to about 99% by weight, preferably from about 90 to about 98% by weight, of acrylic acid, at least one non-ethoxylated ester of acrylic acid with linear C10-C30 monoalcohols in a total amount of about 0.9-19.9% by weight, preferably about 2-10% by weight, and at least one crosslinking agent in a total amount of about 0.1-4% by weight.

The crosslinked copolymers claimed in the present disclosure, built up from acrylic acid and non-ethoxylated esters of acrylic acid with linear C10-C30 monoalcohols as monomers, are amphiphilic copolymers.

Preferred crosslinked copolymers based on acrylic acid and non-ethoxylated esters of acrylic acid with linear C10-C30 monoalcohols as monomers are the following commercial products of Lubrizol Advanced Materials, Inc: Carbopol ETD 2020 Polymer, Carbopol 1342 Polymer, Carbopol 1382 Polymer, Carbopol SC 200, Carbopol SC 500 Polymer, Carbopol Ultrez 20 Polymer, Carbopol Ultrez 21 Polymer, Carbopol Xtra-11 Polymer, Pemulen EZ-4U Polymer-Emulgator, Pemulen TR-1 Polymer and Pemulen TR-2 Polymer. Other copolymers suitable for the present disclosure, built up from acrylic acid and non-ethoxylated esters of acrylic acid with linear C10-C30 monoalcohols as monomers, are the commercial products Acritamer 501 ED (Rita Corp.), Acritamer 505 ED (Rita Corp.), from Sumitomo Seika Chemicals Co., Ltd.: Aqupec HV-701EDR, Aqupec HV-501ER, Aqupec SER W-150C and Aqupec SER W-300C, also Tego Carbomer 341 ER and TEGO Carbomer 750 HD, both from Evonik Nutrition & Care GmbH.

The commercial product Ultrez 21 from Lubrizol Advanced Materials with the INCI designation Acrylates/C10-30 Alkyl Acrylates Crosspolymer is an extremely preferred crosslinked copolymer as contemplated herein, based on acrylic acid and non-ethoxylated esters of acrylic acid with linear C10-C30 monoalcohols as monomers.

Alkalizing agent up to a pH value of the cosmetic composition in the range about 5-8, preferably in the range about 6-7, particularly preferably in the range about 6.2-6.8, each measured at about 20° C.

In order to neutralize or, as the case may be, to neutralize the cross-linked copolymer, built up from acrylic acid and non-ethoxylated esters of acrylic acid with linear C10-C30 monoalcohols as monomers in order for the thickening effect to occur, the cosmetic compositions as contemplated herein further contain at least one alkalizing agent in an amount such that the pH of the cosmetic composition is in the range of about 5-8, preferably in the range of about 6-7, particularly preferably in the range of about 6.2-6.8, in each case measured at about 20° C.

Alkalizing agents are selected from the group comprising alkanolamines and alkali metal hydroxides and mixtures thereof. Preferably lithium, sodium, potassium, especially sodium or potassium, serve as alkali metal ions of the mentioned hydroxides.

As contemplated herein, to obtain a carrier medium that is as transparent as possible, it is preferred that the cosmetic compositions contain at least one alkanolamine as alkalizing agent. The alkanolamines which can be used as alkalizing agents are preferably selected from primary amines with a C₂-C₆ alkyl base body which carries at least one hydroxyl group. Alkanolamines of particular preference are selected from 2-amino-2-methylpropan-1-ol (AMP), triethanolamine, triisopropanolamines (1,1′,1″-nitrilotris-2-propanol), tromethamines (2-amino-2-(hydroxymethyl)-1,3-propanediol), tetrahydroxypropyl ethylenediamines (CAS number 102-60-3), 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol and 2-amino-2-methylpropan-1,3-diol and mixtures thereof. Alkanolamines particularly preferred as contemplated herein are selected from 2-amino-2-methylpropan-1-ol (AMP), triethanolamine, triisopropanolamines (1,1′,1″-nitrilotris-2-propanol), tromethamine (2-amino-2-(hydroxymethyl)- 1,3-propanediol), tetrahydroxypropyl ethylenediamine, 2-aminoethan-1-ol, and 2-amino-2-methyl-propan-1,3-diol as well as mixtures thereof. As contemplated herein, an extremely preferred alkanolamine is 2-amino-2-methylpropan-1-ol.

In order to achieve a carrier medium that is as transparent as possible, which can be sprayed with a pump sprayer, but still has a sufficiently high viscosity so that a total pigment content of about 0.1-10 wt.-% by weight, based on the weight of the cosmetic composition, can be dispersed in a storage-stable manner, as contemplated herein, preferred compositions contain, in each case based on their weight, at least one alkanolamine selected from 2-amino-2-methylpropan-1-ol (AMP), triethanolamine, triisopropanolamines (1,1′,1″-nitrilotris-2-propanol), tromethamines (2-amino-2-(hydroxymethyl)-1,3-propanediol), tetrahydroxypropyl ethylenediamines, 2-aminoethan-1-ol and 2-amino-2-methyl-propan-1,3-diol and mixtures thereof, in a total amount of about 0.05-0.5 wt.-%, preferably about 0.10-0.40% by weight, more preferably about 0.15-0.30% by weight and particularly preferably about 0.20-0.25% by weight.

In order to achieve a carrier medium that is as transparent as possible, which can be sprayed with a pump sprayer, but still has a sufficiently high viscosity so that a total pigment content of about 0.1-10 wt.-%, based on the weight of the cosmetic composition, can be dispersed in a storage-stable manner, as contemplated herein, extremely preferred compositions contain, in each case based on their weight, about 0.05-0.5% by weight, preferably about 0.10-0.40% by weight, more preferably about 0.15-0.30% by weight and particularly preferably from about 0.20 to about 0.25% by weight of 2-amino-2-methylpropan-1-ol.

Provided that the ethanol content of the cosmetic compositions as contemplated herein is at the claimed lower limit, i.e. in the range of about 30-45% by weight of ethanol, based on the weight of the cosmetic composition, alkali metal hydroxides, in particular sodium hydroxide and/or potassium hydroxide, may be contained as sole alkalizing agents.

The alkali hydroxides which can be used as alkalizing agents are preferably selected from sodium hydroxide and potassium hydroxide and mixtures thereof.

Cosmetic compositions preferred as contemplated herein contain, in each case based on their weight, about 30-45% by weight of ethanol and at least one alkalizing agent selected from potassium hydroxide and sodium hydroxide and mixtures thereof, in a total amount of about 0.05-0.5% by weight, preferably of about 0.10-0.40% by weight, more preferably of about 0.15-0.30% by weight and particularly preferably of about 0.20-0.25% by weight, in each case based on the weight of the cosmetic composition.

### Oils and Fragrances

Cosmetic compositions as contemplated herein at least one substance selected from oils and fragrances is contained in a total amount of about 0-10% by weight, preferably about 0.5-8% by weight, particularly preferably about 1-6% by weight, extremely preferably about 2-5% by weight, further extremely preferably about 3-4% by weight, each based on the weight of the cosmetic composition.

The cosmetic oil is liquid under normal conditions (about 20° C., about 1013.25 mbar); essential oils and perfume oils or fragrances are not counted as cosmetic oils. Cosmetic oils which are liquid under normal conditions are not miscible with water.

As contemplated herein, essential oils are mixtures of volatile components produced by steam distillation from vegetable raw materials, e.g. citrus oils.

In so far as the present application refers to a cosmetic oil, it is always a cosmetic oil which is neither a perfume nor an essential oil, is liquid under normal conditions and is not miscible with water.

The definition of a fragrance within the meaning of the present notification is in line with the usual professional definition as it can be found in the ROMPP Chemie Lexikon, December 2007. According to this, a fragrance is a chemical compound with smell and/or taste that excites the receptors of the hair cells of the olfactory system (adequate stimulus). The physical and chemical properties required for this are a low molar mass of maximum about 300 g/mol, a high vapor pressure, minimal water, and high lipid solubility as well as weak polarity and the presence of at least one osmophoric group in the molecule. To distinguish volatile, low-molecular substances which are normally, and also for the purposes of the present application, not considered and used as perfume but primarily as solvents, such as ethanol, propanol, isopropanol and acetone, from perfumes of the present disclosure, perfumes of the present disclosure have a molecular weight of from about 74 to about 300 g/mol, contain at least one osmophoric group in the molecule and have an odor and/or taste, that is to say, they excite the receptors of the hair cells of the olfactory system.

Since cosmetic compositions are usually perfumed, as contemplated herein, preferred cosmetic compositions contain at least one perfuming agent in a total amount of about 0.05-7% by weight, preferably about 0.1-5% by weight, particularly preferably about 0.5-3% by weight, extremely preferably about 1-2% by weight, each based on the weight of the cosmetic composition.

Examples of fragrance and odoriferous compounds of the ester type preferred by the present disclosure are benzyl acetate, phenoxyethyl isobutyrate, p-tert. butyl cyclohexyl acetate, linalyl acetate, dimethyl benzyl carbonyl acetate (DMBCA), phenyl ethyl acetate, benzyl acetate, ethyl methyl phenyl glycinate, allyl cyclohexyl propionate, styrene allyl propionate, benzyl salicylate, cyclohexyl salicylate, floramate, melusate and jasmecyclate. Examples of fragrance and odoriferous compounds of the ether type preferred as contemplated herein are benzylethyl ether and ambroxane, examples of fragrance and odoriferous compounds of the aldehyde type preferred as contemplated herein are the linear alkanals with 8-18 C atoms, citral, citronellal, citronellyloxy-acetaldehyde, cyclamenaldehyde, lilyal and bourgeonal, Examples of odoriferous compounds of the ketone type preferred by the present disclosure are jonones, alpha-isomethylionone and methylcedryl ketone; examples of odoriferous compounds of the alcohol type preferred by the present disclosure are anethole, citronellol, eugenol, geraniol, linalool, phenylethyl alcohol and terpineol; examples of odoriferous compounds of the terpene type preferred by the present disclosure are limonene and pinene. Examples of fragrance and scent compounds preferred by the present disclosure are pine, citrus, jasmine, patchouli, rose, ylang-ylang oil, muscatel sage oil, camomile oil, clove oil, mint oil, cinnamon leaf oil, lime blossom oil, juniper berry oil, vetiver oil, olibanum oil, galbanum oil, labdanum oil, orange blossom oil, neroli oil, Orange peel oil and sandalwood oil, furthermore the essential oils like angelica root oil, anise oil, arnica flower oil, basil oil, bay oil, bergamot oil, champaca flower oil, silver fir oil, silver fir cone oil, elemi oil, eucalyptus oil, fennel oil, spruce needle oil, geranium oil, ginger grass oil, guaiac wood oil, gurjun balsam oil, helichrysum oil, Ho oil, Ginger oil, iris oil, cajeput oil, calamus oil, camomile oil, camphor oil, canaga oil, cardamom oil, cassia oil, pine needle oil, copaïva balsam oil, coriander oil, spearmint oil, Cumin oil, cumin oil, lavender oil, lemongrass oil, lime oil, mandarin oil, lemon balm oil, musk seed oil, myrrh oil, clove oil, niaouli oil, orange oil, origanum oil, Palmarosa oil, patchouli oil, Peru balsam oil, petitgrain oil, pepper oil, peppermint oil, pimento oil, pine oil, rose oil, rosemary oil, sandalwood oil, celery oil, spik oil, star anise oil, turpentine oil, thuja oil, thyme oil, verbena oil, juniper berry oil, wormwood oil, wintergreen oil, hyssop oil, cinnamon oil, citronella oil, lemon oil and cypress oil. Other fragrance and aroma compounds are ambrettolide, alpha-amyl cinnamaldehyde, anethole, anisaldehyde, anise alcohol, anisole, methyl anthranilic acid ester, acetophenone, benzyl acetone, Benzaldehyde, ethyl benzoate, benzophenone, benzyl alcohol, benzyl acetate, benzyl benzoate, benzyl formate, benzyl valerianate, borneol, bornyl acetate, a-bromostyrene, n-decylaldehyde n-dodecylaldehyde, eugenol, eugenol methyl ether, eucalyptol, farnesol, fenchone, fenchyl acetate, geranyl acetate, geranyl formate, heliotropin, methyl heptanecarboxylate, heptaldehyde, Hydroquinone dimethyl ether, hydroxycinnamic aldehyde, hydroxycinnamic alcohol, indole, iron, isoeugenol, isoeugenol methyl ether, isosafrol, jasmone, camphor, carvacrol, carvone, p-cresol methyl ether, coumarin, p-methoxyacetophenone, methyl-n-amylketone, methyl anthranilic acid methyl ester, p-methylacetophenone, methyl chavicol, p-methylquinoline, methyl-b-naphthylketone, methyl-n-nonyl acetaldehyde, Methyl-n-nonylketone, muscone, beta-naphthol ethyl ether, beta-naphthol methyl ether, nerol, nitrobenzene, n-nonylaldehyde, nonyl alcohol, n-octylaldehyde, p-oxy-acetophenone, pentadecanolide, beta-phenylethyl alcohol, phenylacetaldehyde dimethyacetal, phenylacetic acid, pulegon, safrole, isoamyl salicylic acid ester, methyl salicylic acid ester, hexyl salicylic acid ester, cyclohexyl salicylic acid ester, Santalol, skatole, terpineol, thym, thymol, gamma-undecalactone, vanillin, veratrum aldehyde, cinnamic aldehyde, cinnamic alcohol, cinnamic acid, ethyl cinnamate and benzyl cinnamate.

Other also preferred (more volatile) fragrances are alkyl isothiocyanates (alkyl legumes), butanedione, limonene, linalool, linayl acetate and propionate, menthol, menthone, methyl-n-heptenone, phellandrene, phenylacetaldehyde, terpinyl acetate, citral and citronellal.

Preferably, the cosmetic compositions preferred by the present disclosure contain a mixture of different fragrances which together create an appealing scent.

Suitable perfume oils may also contain natural fragrance mixtures as available from plant or animal sources, e.g. pine, citrus, jasmine, rose, lily, or ylang-ylang oil. Essential oils of lower volatility, which are mostly used as aromatic components, are also suitable as perfume oils, e.g. sage oil, camomile oil, melissa oil, mint oil, cinnamon leaf oil, lime blossom oil, juniper berry oil, vetiver oil, olive oil, galbanum oil, laudanum oil, clove oil, iso-eugenol, thyme oil, bergamot oil, geranium oil and rose oil.

Surprisingly, it was found that a low content of at least one cosmetic oil can improve the adhesion of the pigments to the treated hair or skin. Cosmetic compositions preferred as contemplated herein contain at least cosmetic oil in a total amount of about 0.05-5% by weight, preferably about 0.1-4% by weight, particularly preferably about 0.5-3% by weight, extremely preferably about 1-2% by weight, each based on the weight of the cosmetic composition.

As contemplated herein, particularly preferred cosmetic oils are selected from the esters of linear or branched saturated or unsaturated fatty alcohols with 2-30 carbon atoms with linear or branched saturated or unsaturated fatty acids with 2-30 carbon atoms, which may be hydroxylated. These include cetyl 2-ethylhexanoate, 2-hexyl decyl stearate, 2-hexyl decyl laurate, isodecyl neopentanoate, isononylisononanoate, 2-ethylhexyl palmitate and 2-ethylhexyl stearate. Also preferred are isopropyl myristate, isopropyl palmitate, isopropyl stearate, isopropyl isostearate, isopropyl oleate, isooctyl stearate, Isononylstearat, isocetyl stearate, isononyl isononanoate, isotridecyl isononanoate, cetearyl isononanoate, 2-Etthylhexyl laurate, 2-ethylhexyl, 2-ethylhexyl cocoate, 2-octyldodecyl palmitate, butyl octanoic acid-2-butyl octanoate, Diisotridecylacetat, n-Butyl stearate, n-Hexyl laurate, n-Decyl oleate, oleyl oleate, olerlerucate, erucyl oleate, erucylerucate, ethylene glycol dioleate and ethylene glycol dipalmitate.

Other cosmetic oils favored by the present disclosure are selected from natural and synthetic hydrocarbons, especially preferably mineral oils, paraffin oils, C₁₈-C₃₀ isoparaffins, especially isoeicosan, polyisobutenes and polydecenes, which are for example marketed under the name Emery® 3004, 3006, 3010 or available under the designation Ethylflo® from Albemarle or Nexbase® 2004G from Nestle, further selected from C8-C16 isoparaffins, in particular from isodecane, isodododecane, isotetradecane and isohexadecane as well as mixtures thereof, and 1,3-di(2-ethylhexyl)-cyclohexane.

Other cosmetic oils favored by the present disclosure are selected from the benzoic acid esters of linear or branched C8-22 alkanols. C12-C15-alkyl benzoate, isostearyl benzoate, ethylhexyl benzoate and octyl docecyl benzoate are particularly preferred.

Other cosmetic oils preferred by the present disclosure are selected from fatty alcohols with 6-30 carbon atoms which are unsaturated or branched and saturated or branched and unsaturated. The branched alcohols are often referred to as Guerbet alcohols because they are available after the Guerbet reaction. Preferred alcohol oils are 2-hexyldecanol, 2-octyldodecanol, 2-ethylhexyl alcohol and isostearyl alcohol.

Other cosmetic oils preferred by the present disclosure are selected from mixtures of Guerbet alcohols and Guerbet alcohol esters, e.g. mixtures of 2-hexyl decanol and 2-hexyl decyl laurate.

Further cosmetic oils preferred as contemplated herein are selected from the triglyceride's (=triple esters of glycerol) of linear or branched, saturated or unsaturated, optionally hydroxylated C8-30-fatty acids. The use of natural oils, e.g. Amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, hazelnut oil, elderberry seed oil, jojoba oil, linseed oil, macadamia nut oil, corn germ oil, almond oil, marula oil, evening primrose oil, olive oil, palm kernel oil, Brazil nut oil, pecan oil, peach kernel oil, rapeseed oil, castor oil, sea buckthorn pulp oil, sea buckthorn oil, sesame oil, soybean oil, sunflower oil, grape seed oil, walnut oil, wild rose oil, wheat germ oil, and the liquid fractions of coconut oil and the likes. However, synthetic triglyceride oils, in particular capric/caprylic triglycerides, such as the commercial products Myritol® 318 or Myritol® 331 (BASF) with unbranched fatty acid residues and glyceryl triisostearin with branched fatty acid residues are also preferred.

Further cosmetic oils which are particularly preferred as contemplated herein are selected from the dicarboxylic acid esters of linear or branched C2-C10-alkanols, in particular diisopropyl adipate, di-n-butyl adipate, di-(2-ethylhexyl) adipate, dioctyl adipate, diethyl/di-n-butyl/Dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further preferred cosmetic oils as contemplated herein are selected from the adducts of 1 to 5 propylene oxide units with mono- or polyvalent C8-22-Alkanols, such as octanol, decanol, decanediol, lauryl alcohol, myristyl alcohol and stearyl alcohol, e.g. B. PPG-2 myristyl ether and PPG-3 myristyl ether.

Other cosmetic oils preferred as contemplated herein are selected from the addition products of at least 6 ethylene oxide and/or propylene oxide units to mono- or polyvalent C3-22 alkanols such as glycerol, butanol, butanediol, myristyl alcohol and stearyl alcohol, which may be esterified if desired, e.g. PPG-14-butyl ether, PPG-9-butyl ether, PPG-10-butanediol, PPG-15-stearyl ether and glycereth-7-diisononanoate.

Further preferred cosmetic oils as contemplated herein are selected from the C8-C22 fatty alcohol esters of monovalent or polyvalent C2-C7-hydroxycarboxylic acids, in particular the esters of glycolic acid, lactic acid, malic acid, tartaric acid, citric acid and salicylic acid.

Further cosmetic oils preferred as contemplated herein are selected from the symmetrical, asymmetrical or cyclic esters of carbonic acid with C₃₋₂₂-alkanols, C₃₋₂₂-alkanediols or C₃₋₂₂-alkanetriols, e.g. B. dicaprylyl carbonate or the esters according to the teaching of DE 19756454 A1, in particular glycerol carbonate.

Further cosmetic oils, which may be preferred as contemplated herein, are selected from the esters of dimers of unsaturated C₁₂-C₂₂-fatty acids (dimer fatty acids) with monovalent linear, branched or cyclic C₂-C₁₈-Alkanols or with polyvalent linear or branched C2-C6-Alkanols.

Further cosmetic oils which are suitable as contemplated herein are selected from the silicone oils, to which e.g. Dialkyl- and alkyl aryl siloxanes, such as cyclopentasiloxane, cyclohexasiloxane, dimethylpolysiloxane and methylphenylpolysiloxane, but also hexamethyldisiloxane, octamethyltrisiloxane and decamethyltetrasiloxane count. Preferred can be volatile silicone oils, which can be cyclic, such as octamethylcyclotetrasiloxane, decamethylcyclopentasiloxane and dodecamethylcyclohexasiloxane, as well as mixtures thereof, as described, for. B. are contained in the commercial products DC 244, 245, 344 and 345 from Dow Corning. Also suitable are volatile linear silicone oils, hexamethyldisiloxane (L₂), Octamethyltrisiloxane (L₃), Decamethyltetrasiloxane (L₄) and any two- and three-component mixtures of L₂, L₃ and/or L₄, preferably mixtures such as those described, for. B. in the commercial products DC 2-1184, Dow Corning® 200 (0.65 cSt) and Dow Corning® 200 (1.5 cSt) from Dow Corning. Preferred non-volatile silicone fluids are selected from higher molecular weight linear dimethylpolysiloxanes, commercially available e.g. under the designation Dow Corning® 190, Dow Corning® 200 Fluid with kinematic viscosities (25° C.) in the range of 5-100 cSt, preferably 5-50 cSt or also 5-10 cSt, and dimethylpolysiloxane with a kinematic viscosity (25° C.) of about 350 cSt.

As contemplated herein, it can be extremely preferred to use mixtures of the oils.

Preferred cosmetic compositions as contemplated herein, contain at least one cosmetic oil selected from natural and synthetic hydrocarbons, particularly preferably paraffin oils, C₁₈-C₃₀ isoparaffins, in particular isoeicosane, polyisobutenes and polydecenes, C₈-C₁₆ isoparaffins, and 1,3-di(2-ethylhexyl)cyclohexane; the benzoic acid esters of linear or branched C₈₋₂₂ alkanols; fatty alcohols with 6-30 carbon atoms, unsaturated or branched and saturated or branched and unsaturated; triglycerides of linear or branched, saturated or unsaturated, optionally hydroxylated C₈₋₃₀-fatty acids, in particular natural oils; the dicarboxylic acid esters of linear or branched C₂-C₁₀ alkanols; the esters of linear or branched, saturated or unsaturated fatty alcohols with 2-30 carbon atoms with linear or branched, saturated or unsaturated fatty acids with 2-30 carbon atoms which may be hydroxylated; the addition products of 1 to 5 propylene oxide units to mono- or polyvalent C₈₋₂₂-Aalkanols; the addition products of at least 6 ethylene oxide and/or propylene oxide units to mono- or polyvalent C₃₋₂₂ alkanols; the C₈-C₂₂ fatty alcohol esters of monovalent or polyvalent C₂-C₇ hydroxycarboxylic acids; the symmetrical, asymmetrical or cyclic esters of carbonic acid with C₃₋₂₂ alkanols, C₃₋₂₂ alkanediols or C₃₋₂₂ alkanetriols; the esters of dimers of unsaturated C₁₂-C₂₂-fatty acids (dimer fatty acids) with monovalent linear, branched or cyclic C2-C18-alkanols or with polyvalent linear or branched C₂-C₆-alkanols; silicone oils and mixtures of the above substances, in a total amount of about 0.05-5% by weight-%, preferably about 0.1-4 wt. %, particularly preferably about 0.5-3 wt. %, extremely preferably about 1-2 wt. %, each based on the weight of the cosmetic composition.

### Surfactants

Since the cosmetic compositions as contemplated herein have a considerable ethanol content, the hydrophobic fragrances, and oils in the claimed total amount of up to 10% by weight can be easily incorporated without higher surfactant amounts. The cosmetic compositions as contemplated herein at least one surfactant is contained in a total amount of about 0-0.1% by weight, preferably about 0-0.05% by weight, particularly preferably about 0-0.01% by weight, each based on the weight of the cosmetic composition.

For the purposes of the present application, surfactants and emulsifiers are amphiphilic (bifunctional) compounds which include at least one hydrophobic and at least one hydrophilic part of the molecule. The hydrophobic radical is preferably a hydrocarbon chain with 8-28 carbon atoms, which can be saturated or unsaturated, linear, or branched. This C₈-C₂₈-Alkyl chain is particularly preferably linear. Basic properties of surfactants and emulsifiers are the oriented absorption at interfaces as well as the aggregation to micelles and the formation of lyotropic phases.

The cross-linked copolymers used as contemplated herein, composed of acrylic acid and non-ethoxylated esters of acrylic acid with linear C10-C30 monoalcohols as monomers, are not capable of forming micelles or lyotropic phases and therefore do not constitute surfactants or emulsifiers within the meaning of the present application.

As contemplated herein, anionic, non-ionic, cationic, zwitterionic and amphoteric surfactants are suitable.

All anionic surfactants suitable for use on the human body are suitable as anionic surfactants in the compositions as contemplated herein. These are exemplified by a water-solubilizing anionic group such as a carboxylate, sulphate, sulphonate or phosphate group and a lipophilic alkyl group with 8 to 30 C atoms. In addition, glycol or polyglycol ether groups, ester, ether and amide and hydroxyl groups may also be present in the molecule. Examples of suitable anionic surfactants are linear and branched fatty acids with 8 to 30 C atoms (soaps), alkyl ether carboxylic acids, acyl sarcosinates, acyl taurates, acyl isethionates, sulphosuccinic acid monoalkyl esters, dialkyl sulfosuccinates and monoalkyl polyoxyethyl sulfosuccinates, linear alkanesulfonates, linear alpha-olefin sulfonates, alkyl sulfates and alkyl ether sulfates and alkyl and/or alkenyl phosphates. Examples of such surfactants are the compounds with the INCI designations sodium laureth sulphates, sodium lauryl sulphates, sodium myreth sulphates or sodium laureth carboxylates.

Zwitterionic surfactants are surface-active compounds which carry at least one quaternary ammonium group and at least one carboxylate, sulphonate or sulphate group in the molecule. Examples of zwitterionic surfactants are the so-called betaines such as the N-alkyl-N,N-dimethylammonium glycinate, for example the cocoalkyl dimethylammonium glycinate, N-acylaminopropyl-N,N-dimethylammonium glycinate, for example, cocoacylaminopropyl dimethyl ammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate.

Amphoteric surfactants are surface-active compounds which, apart from a C₈-C₂₄ alkyl or acyl group, contain at least one free amino group and at least one —COOH or —SO₃H group in the molecule and can form internal salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycine, N-alkyltaurines, N-alkylsarcosines, 2-alkylaminopropionic acids and alkylaminoacetic acids each with 8 to 24 C atoms in the alkyl group.

Nonionic surfactants contain e.g. a polyol group, a polyalkylene glycol ether group or a combination of polyol and polyglycol ether group as hydrophilic group. Such compounds are, for example, addition products of 4 to 50 mol ethylene oxide and/or 0 to 5 mol propylene oxide to linear and branched fatty alcohols, to fatty acids and to alkylphenols, each with 8 to 20 C atoms in the alkyl group, ethoxylated mono-, di- and triglycerides, such as glycerol monolaurate+20 ethylene oxide, and glycerol monostearate+20 ethylene oxide, sorbitan fatty acid esters and adducts of ethylene oxide with sorbitan fatty acid esters such as polysorbates (Tween 20, Tween 21, Tween 60, Tween 61, Tween 81), adducts of ethylene oxide with fatty acid alkanolamides and fatty amines, and alkyl polyglycosides.

### Pigment

The cosmetic compositions as contemplated herein at least one pigment is contained in a total amount of about 0.1-10% by weight, preferably about 0.2-8% by weight, particularly preferably about 1-6% by weight, extremely preferably about 2-5% by weight, further extremely preferably about 3-4% by weight, each based on the weight of the cosmetic composition.

Pigments within the meaning of the present disclosure are coloring compounds which have a solubility in water at about 25° C. of less than about 0.5 g/L, preferably less than about 0.1 g/L, still more preferably less than about 0.05 g/L. Water solubility can be determined, for example, by the method described below: about 0.5 g of the pigment are weighed in a beaker. A stir-fish is added. Then one liter of distilled water is added. This mixture is heated to about 25° C. for one hour while stirring on a magnetic stirrer. If undissolved components of the pigment are still visible in the mixture after this period, the solubility of the pigment is below about 0.5 g/L. If the pigment-water mixture cannot be assessed visually due to the high color intensity of the finely dispersed pigment, the mixture is filtered. If a proportion of undissolved pigments remains on the filter paper, the solubility of the pigment is below about 0.5 g/L.

Pigments suitable for present disclosure can be of inorganic and/or organic origin.

Due to their excellent light, weather and/or temperature resistance, the use of inorganic pigments is particularly preferred in cosmetic compositions as contemplated herein.

The preferred number-average particle size of the—preferably inorganic—pigments is from about 0.1 μm to about 1 mm, more preferably from about 0.5 μm to about 750 μm and especially from about 10 μm to about 500 μm.

In a preferred embodiment, compositions as contemplated herein at least one pigment is selected from inorganic pigments.

Pigments preferred by present disclosure are selected from synthetic or natural inorganic pigments. Inorganic pigments of natural origin can be produced, for example, from chalk, ochre, umber, green earth, fired Terra di Siena or graphite. Other inorganic pigments preferred as contemplated herein are selected from white pigments, such as titanium dioxide, especially rutile, anatase or brookite, black pigments, such as iron oxide black, colored pigments, such as ultramarine or iron oxide red, as well as fluorescent or phosphorescent pigments and mixtures of these pigments.

Inorganic pigments particularly preferred as contemplated herein are selected from colored metal oxides, colored metal hydroxides and colored metal oxide hydrates, mixed phase pigments, sulfur-containing silicates, silicates, metal sulfides, complex metal cyanides, metal sulphates, metal chromates and/or metal molybdates. Especially preferred pigments are titanium dioxide (CI 77891), black iron oxide (CI 77499), yellow iron oxide (CI 77492), red and brown iron oxide (CI 77491), manganese violet (CI 77742), Ultramarine (sodium aluminum sulfosilicates, CI 77007, Pigment Blue 29), chromium oxide hydrate (CI77289), iron blue (ferric ferrocyanides, CI77510) and/or Carmine (Cochineal).

Other pigments preferred by the inventors are the so-called pearlescent pigments. These usually have a pigment core of a platelet-shaped material, preferably selected from natural mica, especially muscovite, phlogopite, paragonite, biotite, lepidolite or margarite, and preferably muscovite or phlogopite, synthetic mica, and borosilicates, in particular sodium borosilicate, calcium borosilicate, calcium aluminum borosilicate and calcium sodium borosilicate, the pigment core being coated with one or more layers comprising one or more of the aforementioned metal oxides or a metal oxychloride, such as bismuth oxychloride. Such borosilicate platelets are often referred to as glass particles.

As an alternative to natural mica, synthetic mica coated with one or more metal oxide(s) can also be used as a pearlescent pigment. Such pearlescent pigments based on synthetic mica, which are suitable as contemplated herein, are described in the disclosure document WO 2005/065632, to which explicit reference is made.

The color of the respective pigments is varied by varying the layer thickness of the metal oxide or oxides and by the qualitative selection of the metal oxide or oxides.

Examples of pearlescent pigments that are particularly suitable as contemplated herein are commercially available under the trade names Timiron®, Rona®, Xirona®, Colorona® and Dichrona® from Merck, Ariabel® from Sensient, Prestige® or Mirage® from Eckart Cosmetic Colors, Sunshine® from Sunstar and Oxen from Hebei Oxen New Materials.

In a preferred form, the cosmetic composition as contemplated herein contains as pigment at least one color, effect and/or pearlescent pigment selected from metal oxides, metal hydroxides, hydrated metal oxides, sulfur-containing silicates, metal sulfides, complex metal cyanides, metal sulphates, bronze pigments and/or pearlescent pigments, the latter comprising a pigment core of a platelet-shaped material, which is selected from natural mica (mica), in particular muscovite, phlogopite, paragonite, biotite, lepidolite or margarite and preferably muscovite or phlogopite, from synthetic mica, and from borosilicates, in particular sodium borosilicate, calcium borosilicate, calcium aluminum borosilicate and calcium sodium borosilicate, wherein the pigment core may be coated with one or more layers of one or more metal oxides or a metal oxychloride. Especially the platelet-shaped pigment cores of natural or synthetic mica are preferably coated with one or more layers of one or more metal oxides or a metal oxychloride. The platelet-shaped pigments of borosilicates are especially preferred uncoated.

Especially preferred are cosmetic compositions as contemplated herein containing one or more borosilicate or mica pigments which are combined with titanium dioxide (CI 77891), manganese violet (CI 77742), black iron oxide (CI 77499), yellow iron oxide (CI 77492), may be coated with red and/or brown iron oxide (CI 77491, CI 77499), and/or ultramarine (sodium aluminum sulfosilicates, CI 77007, pigment blue 29), chromium oxide hydrate (CI 77289), chromium oxide (CI 77288) and/or iron blue (ferric ferrocyanides, CI 77510). Especially the platelet-shaped pigment cores of natural or synthetic mica are preferably coated with one or more layers of one or more metal oxides or a metal oxychloride. The platelet-shaped pigments of borosilicates are especially preferred uncoated.

For the selection of pigments that are particularly preferred as contemplated herein, the requirement to use only titanium dioxide free pigments still applies, as the use of pigments containing titanium dioxide in cosmetics is now subject to certain restrictions in some countries or will be subject to restrictions in the near future.

In a further particularly preferred embodiment, the cosmetic composition as contemplated herein therefore contains as inorganic pigment at least one titanium dioxide-free color, effect and/or pearlescent pigment selected from metal oxides with the exception of titanium dioxide, metal hydroxides, metal oxide hydrates, sulfur-containing silicates, metal sulfides, complex metal cyanides, metal sulphates, bronze pigments and/or pearlescent pigments, the latter comprising a pigment core of a platelet-shaped material, which is selected from natural mica (mica), in particular muscovite, phlogopite, paragonite, biotite, lepidolite or margarite and preferably muscovite or phlogopite, from synthetic mica, and from borosilicates, in particular sodium borosilicate, calcium borosilicate, calcium aluminum borosilicate and calcium sodium borosilicate, wherein the pigment core may be coated with one or more layers of one or more metal oxides, titanium dioxide excluded, or a metal oxychloride. Especially preferred are cosmetic compositions as contemplated herein, which contain one or more titanium dioxide free borosilicate or mica pigments containing manganese violet (CI 77742), black iron oxide (CI 77499), yellow iron oxide (CI 77492), may be coated with red and/or brown iron oxide (CI 77491, CI 77499), and/or ultramarine (sodium aluminum sulfosilicates, CI 77007, pigment blue 29), chromium oxide hydrate (CI 77289), chromium oxide (CI 77288) and/or iron blue (ferric ferrocyanides, CI 77510).

As an alternative to natural mica, synthetic natural mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (Titanium Dioxide)

### Colorona RY, Merck, CI 77891 (Titanium Dioxide), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (Titanium Dioxide)

### Colorona Blackstar Blue, Merck, CI 77499 (IRON OXIDES), MICA

### Colorona Patagonian Purple, Merck, MICA, CI 77491 (Iron Oxides), CI 77891 (Titanium Dioxide), CI 77510 (FERRIC FERROCYANIDE)

### Colorona Red Brown, Merck, MICA, CI 77491 (Iron Oxides), CI 77891 (Titanium Dioxide)

### Colorona Russet, Merck, CI 77491 (Titanium Dioxide), MICA, CI 77891 (Iron Oxides)

### Colorona Imperial Red, Merck, MICA, Titanium Dioxide (CI 77891), D&C RED NO. 30 (CI 73360)

### Colorona Majestic Green, Merck, CI 77891 (Titanium Dioxide), MICA, CI 77288 (Chromium Oxide Greens)

### Colorona Light Blue, Merck, MICA, Titanium Dioxide (CI 77891), Ferric Ferrocyanide (CI 77510)

### Colorona Red Gold, Merck, MICA, CI 77891 (Titanium Dioxide), CI 77491 (Iron Oxides)

### Colorona Gold Plus MP 25, Merck, MICA, Titanium Dioxide (CI 77891), Iron Oxides (CI 77491)

### Colorona Carmine Red, Merck, MICA, Titanium Dioxide, Carmine

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (Titanium Dioxide), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, Mica, Titanium Dioxide (CI 77891), Iron Oxides (CI 77491)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona® Golden Sky, Merck, Silica, CI 77891 (Titanium Dioxide), Tin Oxide

### Xirona® Caribbean Blue, Merck, Mica, CI 77891 (Titanium Dioxide), Silica, Tin Oxide

### Xirona® Kiwi Rose, Merck, Silica, CI 77891 (Titanium Dioxide), Tin Oxide

### Xirona® Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment, the cosmetic composition as contemplated herein may also contain one or more coloring compounds from the group of organic pigments.

The organic pigments as contemplated herein are correspondingly insoluble, organic dyestuffs or color lacquers, which can be selected, for example, from the group of nitroso, nitro-azo, xanthene, anthraquinone, isoindolinone, isoindolinone, quinacridone, perinone, perylene, diketo-pyrrolopyrrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470 may be included.

The organic pigment can also be a color paint. For the purposes of the present disclosure, the term “color paint” is understood to mean particles comprising a layer of absorbed dyes, the unit of particle and dye being insoluble under the above conditions. The particles can, for example, be inorganic substrates, which can be silica, calcium borosilate, calcium aluminum borosilicate or even aluminum. For example, alizarin color varnish can be used.

Due to their excellent light and temperature resistance, the use of the pigments in cosmetic compositions as contemplated herein is particularly preferred. It is also preferred if the pigments used have a certain particle size. This particle size leads on the one hand to an even distribution of the pigments when applied to the hair or skin and on the other hand avoids a rough hair or skin feeling after application of the cosmetic composition. As contemplated herein, it is therefore preferred if the at least one pigment has an average particle size D₅₀ of from about 1.0 to about 50 μm, preferably of from about 5.0 to about 45 μm, preferably of from about 10 to about 40 μm, in particular of from about 14 to about 30 μm. The average particle size D₅₀, for example, can be determined by dynamic light scattering (DLS).

The size of the substrate platelet can be adjusted to the respective application purpose, especially the desired effect on the keratinic material. As a rule, the substrate platelets have a number average largest diameter of from about 2 to about 200 μm, preferably from about 5 to about 100 μm, especially preferably from about 10 to about 40 μm.

In a preferred design, the aspect ratio, expressed by the ratio of the average size to the average thickness, is at least about 80, preferably at least about 200, more preferably at least about 500, more preferably more than about 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for about 3 minutes.

Due to their irregular structure, pigments based on lamellar substrate platelets generate a high proportion of scattered light. Furthermore, pigments based on lamellar substrate platelets do not completely cover the existing color of the treated keratinous material, i.e. hair or skin, and effects like natural hair greying can be achieved, for example.

Lenticular (=lenticular) substrate platelets have an essentially regular round edge and are also known as “silver dollars” due to their appearance. Due to their regular structure, the proportion of reflected light predominates in pigments based on lenticular substrate platelets.

The adhesion and abrasion resistance of pigments based on lamellar or lenticular substrate platelets on keratinous material such as hair or skin can be significantly increased by modifying the outermost layer with organic compounds such as silanes, phosphoric acid esters, titanates, borates or carboxylic acids. The organic compounds are bound to the surface of the outermost layer, which preferably contains metal oxide. The outermost layer is the layer that is spatially most distant from the lamellar substrate platelet, the pigment core. The organic compounds are preferably functional silane compounds which can bind to the outermost metal oxide containing layer. These can be either mono- or bifunctional compounds. Examples of bifunctional organic compounds are Methacryloxypropenyltrimethoxysilan, 3-Methacryloxypropyltrimethoxysilan, 3-Acryloxypropyltrimethoxysilan, 2-Acryloxyethyltrimethoxysilan, 3-Methacryloxypropyltriethoxysilan, 3-Acryloxypropyltrimethoxysilan, 2-Methacryloxyethyltriethoxysilan, 2-Acryloxyethyltriethoxysilan, 3-Methacryloxypropyltris(methoxyethoxy)silan, 3-Methacryloxypropyltris(butoxyethoxy)silan, 3-Methacryloxypropyltris(propoxy)silan, 3-Methacryloxypropyltris(butoxy)silan, 3-Acryloxypropyltris(methoxyethoxy)silan, 3-Acryloxypropyltris(butoxyethoxy)silan, 3-Acryloxypropyltris(butoxy)silan, Vinyltrimethoxysilan, Vinyltriethoxysilan, Vinylethyldichlorsilan, Vinylmethyldiacetoxysilan, Vinylmethyldichlorsilan, Vinylmethyldiethoxysilan, Vinyltriacetoxysilan, Vinyltrichlorsilan, Phenylvinyldiethoxysilan, or Phenylallyldichlorsilan. Furthermore, a modification with a monofunctional silane, especially with an alkylsilane or an arylsilane, is possible. This has only one functional group that can covalently bond to the surface of the pigment based on coated lamellar substrate platelets (i.e. to the outermost metal oxide-containing layer) or, if not completely covered, to the metal surface. The hydrocarbon residue of the silane points away from the pigment. Depending on the type and nature of the hydrocarbon residue of the silane, a varying degree of hydrophobicity of the pigment is achieved. Examples of such silanes are hexadecyltrimethoxysilane, propyltrimethoxysilane, etc. Pigments based on silica-coated aluminum substrate platelets which are surface modified with a monofunctional silane are particularly preferred. Particularly preferred surface treatment agents for the pigments preferred by the present disclosure are octyltrimethoxysilane, octyltriethoxysilane, hexadecyltrimethoxysilane and hexadecyltriethoxysilane.

Due to the modified surface properties, especially the hydrophobicity, an improvement can be achieved about adhesion, abrasion resistance and alignment in the application.

Other cosmetic compositions favored by the present disclosure contain a pigment based on lamellar and/or lenticular (lenticular) substrate platelets.

### VMP Pigments

Pigments based on a substrate platelet comprising a vacuum metallised pigment (VMP) are also preferred. The substrate platelets of the VMP type have an average thickness of at most about 50 nm, preferably less than about 30 nm, more preferably at most about 25 nm, for example at most about 20 nm. The average thickness of the VMP substrate platelets is at least about 1 nm, preferably at least about 2.5 nm, more preferably at least 5 nm, for example at least about 10 nm. Preferred ranges for the thickness of the VMP substrate platelets are from about 2.5 to about 50 nm, about 5-50 nm, from about 10 to about 50 nm; from about 2.5 to about 30 nm, from about 5 to about 30 nm, from about 10 to about 30 nm; from about 2.5 to about 25 nm, from about 5 to about 25 nm, from about 10 to about 25 nm, from about 2.5 to about 20 nm, from about 5 to about 20 nm and from about 10 to about 20 nm. Preferably, each substrate platelet has a thickness that is as uniform as possible over its entire extent. Due to the small thickness of the substrate platelets, such VMP pigments show a particularly high hiding power.

The substrate platelets of VMP pigments, which are preferred by present disclosure, have a monolithic structure. Monolithic in this context means including a single closed unit without fractures, stratifications, or inclusions, although structural changes may occur within the substrate platelets. The substrate platelets are preferably homogeneously structured, i.e. there is no concentration gradient within the platelets. In particular, the substrate platelets do not have a layered structure and do not have any particles or particles distributed in them. The substrate platelets of preferred VMP pigments can be composed of any material that can be brought into platelet form. They can be of natural origin, but also synthetically produced. Materials of which the VMP substrate platelets may be composed are, for example, metals and metal alloys, metal oxides, preferably aluminum oxide, inorganic compounds, and minerals such as mica and (semi)precious stones, as well as plastics. Preferably the VMP substrate plates are selected from metals or metal alloys. Any metal suitable for metallic lustre pigments can be used. Such metals include iron and steel, as well as all air and water resistant (semi)metals such as platinum, zinc, chromium, molybdenum and silicon, and their alloys such as aluminum bronzes and brass. Preferred metals are aluminum, copper, silver, and gold. Preferred VMP substrate platelets are aluminum platelets and brass platelets, with aluminum (CI 77000) substrate platelets being particularly preferred. Lamellar VMP substrate flakes are exemplified by an irregularly structured edge and are also known as “cornflakes” due to their appearance.

Vacuum metallized pigments (VMP) can be obtained, for example, by releasing metals, metal alloys or metal oxides from appropriately coated films. They are exemplified by a particularly low thickness of the substrate platelets in the range of 5 to 50 nm and a particularly smooth surface with increased reflectivity. In the context of this notification, substrate flakes comprising a pigment metallised under vacuum are also referred to as VMP substrate flakes. VMP substrate platelets of aluminum can be obtained, for example, by releasing aluminum from metallised films.

The metal or metal alloy VMP substrate platelets can be passivated, for example by anodizing (oxide layer) or chromating.

Uncoated lamellar, lenticular and/or VMP substrate plates, especially those made of metal or metal alloy, reflect the incident light to a high degree and create a light-dark flop but no color impression.

A color impression can be created by optical interference effects, for example. Such pigments can be based on at least single-coated substrate platelets. These show interference effects by superimposing differently refracted and reflected light beams. The preferred pigments based on lamellar VMP substrate platelets are for example the pigments of the VISIONAIRE series from Eckart.

Pigments based on lenticular VMP substrate platelets are available under the name Alegrace® Gorgeous from Schlenk Metallic Pigments GmbH.

Pigments based on a substrate platelet comprising a vacuum-metallised pigment are available under the names Alegrace® Marvelous or Alegrace® Aurous from Schlenk Metallic Pigments GmbH, for example.

Other cosmetic compositions favored by the present disclosure are exemplified by the presence of Polyurethane-34. Surprisingly, it was found that a content of Polyurethane-34 can improve the adhesion of the pigments to the treated hair or skin. Polyurethane-34 is a polymer produced in several reaction steps. First, a copolymer of 1,6-hexanediol, neopentylglycol and adipic acid is prepared and reacted with hexamethylene diisocyanate. The resulting polyurethane is reacted with sodium N-(2-aminoethyl)-2-aminoethanesulfonate and ethylenediamine to form the final polymer with the INCI designation Polyurethane-34. A polyurethane-34 preferred by the inventor is marketed by Covestro under the trade name Baycusan® C 1001/1. It has a glass transition temperature of about −51.5° C., measured according to DIN EN 61 006.

In cosmetic compositions particularly preferred as contemplated herein, the content of Polyurethane-34 is about 0.03-1 wt. %, preferably about 0.1-0.7 wt. %, particularly preferably about 0.25-0.4 wt. %, each based on the weight of the cosmetic composition.

A further subject of the present disclosure is a process for the cosmetic treatment of the hair or skin, in which a cosmetic composition according to one of claims 1-10 or according to one of the embodiments described above is applied to the hair or skin, the application preferably being effected by spraying, particularly preferably by spraying with a pump sprayer.

The following examples are intended to illustrate the subject-matter of the present disclosure without limiting it. The quantities in all tables are in weight %, unless otherwise stated.

TABLE 1
Inventive cosmetic compositions (all amounts in % by weight)

| | GO | GO | GO | GO | GO | GO | GO | GO | GO | GO |
|---|---|---|---|---|---|---|---|---|---|---|
| Ingredient | 9\10 | 9\01 | 9\02 | 9\03 | 9\04 | 9\05 | 9\06 | 9\07 | 9\08 | 60\01 |
| Ethanol 96 vol-% | 65.00 | 79.00 | 79.70 | 79.60 | 79.50 | 79.40 | 79.30 | 79.65 | 65.00 | 65.00 |
| denatured (t-butanol, | | | | | | | | | | |
| Bitrex) | | | | | | | | | | |
| demineralized | 32.45 | 19.50 | 19.80 | 19.80 | 19.80 | 19.80 | 19.80 | 19.80 | 34.45 | 25.35 |
| water | | | | | | | | | | |
| Carbopol Ultrez 21 | 0.15 | 0.10 | 0.10 | 0.20 | 0.30 | 0.40 | 0.50 | 0.15 | 0.15 | 0.45 |
| 2-Amino-2-methyl- | 0.20 | 0.90 | 0.20 | 0.20 | 0.20 | 0.20 | 0.20 | 0.20 | 0.20 | 0.20 |
| 1,3-propandiol | | | | | | | | | | |
| Isopropyl myristate | 2.00 | — | — | — | — | — | — | — | — | 2.00 |
| Perfume | — | — | — | — | — | — | — | — | — | 2.00 |
| Timiron Snowflake | 0.20 | 0.50 | 0.20 | 0.20 | 0.20 | 0.20 | 0.20 | 0.20 | 0.20 | — |
| MP-99 | | | | | | | | | | |
| Sodium borosilicate | — | — | — | — | — | — | — | — | — | 5.00 |
| platelets (40-200 μm), | | | | | | | | | | |
| TiO₂-free | | | | | | | | | | |

Even after 6-8 weeks storage at room temperature the pigments remain homogeneously dispersed in the gel.

### Inventive Hair and Skin Treatment

The gels according to Table 1 were filled into a pump spray container. From this, the respective gel was sprayed onto dry hair. The hair showed a beautiful shine with attractive glitter and/or color glitter effects. The pigments adhered well to the hair. The gels as contemplated herein were sprayed onto dry skin in a second treatment process and gave the skin attractive glitter and/or color glitter effects. The pigments adhered well to the skin.

### NON-INVENTIVE EXAMPLES

TABLE 2

| | GO | GO | GO | GO | GO | GO | GO |
|---|---|---|---|---|---|---|---|
| | 197\01 | 197\09 | 197\10 | 197\11 | 197\12 | 197\13 | 197\14 |
| VOLAREST FL-LQ | 1.0 | 1.0 | 1.0 | 1.0 | 1.0 | 1.0 | 2.0 |
| (RB) EN 01955 | | | | | | | |
| Potassium | 0.1 | — | — | — | — | — | — |
| hydroxide 50% | | | | | | | |
| Baycusan C 1001/1 | 1.0 | — | — | — | — | — | — |
| Timiron Snowflake | 0.2 | 0.2 | 0.2 | 0.2 | 0.2 | 0.2 | 0.2 |
| MP-99 | | | | | | | |
| AMP-ULTRA PC 2000 | — | 0.2 | 0.2 | 0.2 | 0.2 | 0.2 | 0.2 |
| Ethanol 96% by | 50.0 | 39.0 | 49.0 | 49.0 | 69.0 | 39.4 | 39.0 |
| volume, denatured | | | | | | | |
| (t-butanol, Bitrex) | | | | | | | |
| Water, | 47.7 | 59.6 | 49.6 | 49.6 | 29.6 | 59.2 | 58.6 |
| demineralized | | | | | | | |

The compositions according to table 2 formed stable gels but could not keep the pigments homogeneously dispersed.

TABLE 3

| | GO | GO | GO | GO | GO | GO | GO | GO |
|---|---|---|---|---|---|---|---|---|
| | 197\01 | 197\02 | 197\03 | 197\04 | 197\05 | 197\06 | 197\07 | 197\08 |
| VOLAREST FL-LQ | 1.0 | 1.0 | 1.0 | 1.0 | 1.00 | 4.0 | 4.0 | 1.0 |
| (RB) EN 01955 | | | | | | | | |
| Potassium | 0.1 | 0.1 | 0.1 | 0.1 | 0.10 | 0.1 | 0.1 | 0.1 |
| hydroxide 50% | | | | | | | | |
| Baycusan C 1001/1 | 1.0 | 1.0 | 1.0 | 1.0 | 1.00 | 1.0 | 1.0 | 1.0 |
| Timiron Snowflake | 0.2 | 0.2 | 0.2 | 0.2 | 0.20 | 0.2 | 0.4 | 0.2 |
| MP-99 | | | | | | | | |
| Glycerin 99.5% | — | — | — | — | 0.40 | — | — | — |
| D-Panthenol 75% | — | — | — | — | 0.15 | — | — | — |
| Bentone 38 V CG | — | — | — | — | 1.57 | — | — | — |
| Perfume | — | — | — | — | 0.08 | — | 0.10 | — |
| Isopropyl myristate | — | — | — | — | 3.15 | — | — | — |
| Ethanol 96% denat. | 50.0 | 60.0 | 70.0 | 80.0 | 64.65 | 70.0 | 70.0 | 39.0 |
| (t-Butanol, Bitrex) | | | | | | | | |
| Water, | 47.7 | 37.7 | 27.7 | 17.7 | 27.70 | 24.7 | 24.4 | 58.7 |
| demineralized | | | | | | | | |

The gels according to table 3 separated and could not keep the pigments homogeneously dispersed.

The test series according to Table 2 and Table 3 show that not every amphiphilic copolymer, here acrylates/beheneth-25 methacrylate copolymer, is suitable for the production of an alcohol-containing gel in which pigments can be homogeneously dispersed with long-term stability.

TABLE 4

| | GO | GO | GO | GO | GO | GO | GO | GO | GO | GO |
|---|---|---|---|---|---|---|---|---|---|---|
| | 144\02 | 144\03 | 144\04 | 144\05 | 144\06 | 144\07 | 144\08 | 144\09 | 144\10 | 144\11 |
| Keltrol CG-SFT | 2.5 | 2.5 | 2.5 | 2.5 | 2.5 | 2.5 | 2.5 | 2.5 | 2.5 | 2.5 |
| Glycerin 99.5 wt.-% | 5.0 | 5.0 | 5.0 | 5.0 | 5.0 | 5.0 | 5.0 | 5.0 | 5.0 | 5.0 |
| Phenoxyethanol | 0.4 | 0.4 | 0.4 | 0.4 | 0.4 | 0.4 | 0.4 | 0.4 | 0.4 | 0.4 |
| Perfume | 0.1 | 0.1 | 0.1 | 0.1 | 0.1 | 0.1 | 0.1 | 0.1 | 0.1 | 0.1 |
| Lactic acid 80% | 0.1 | 0.1 | 0.1 | 0.1 | 0.1 | 0.1 | 0.1 | 0.1 | 0.1 | 0.1 |
| by weight DAB | | | | | | | | | | |
| Dermacryl 79 | 1.0 | — | — | — | — | — | 1.0 | 1.0 | 1.0 | 1.0 |
| Isopropyl | — | 4.0 | 4.0 | 4.0 | 4.0 | 4.0 | — | — | — | — |
| myristate | | | | | | | | | | |
| Colorona | 3.0 | 3.0 | 3.0 | 6.0 | 3.0 | 10.0 | — | 5.0 | — | — |
| Precious Gold | | | | | | | | | | |
| Xirona Golden Sky | — | — | 3.0 | — | 7.0 | — | — | — | — | — |
| Sumicos Walnut | — | — | — | — | — | — | 5.0 | — | — | — |
| 43839 | | | | | | | | | | |
| Prestige Fire Red | — | — | — | — | — | — | — | — | 5.0 | — |
| Oxen C88046 | — | — | — | — | — | — | — | — | — | 5.0 |
| Golden/Kelly/ | | | | | | | | | | |
| Green/Blue | | | | | | | | | | |
| Ethanol 96 Vol.-% | 15.0 | 15.0 | 15.0 | 15.0 | 15.0 | 15.0 | 15.0 | 15.0 | 15.0 | 15.0 |
| denat. (t-Butanol, | | | | | | | | | | |
| Bitrex) | | | | | | | | | | |
| Water, | 72.9 | 69.9 | 66.9 | 66.9 | 62.9 | 62.9 | 70.9 | 70.9 | 70.9 | 70.9 |
| demineralized | | | | | | | | | | |

The gels listed in table 4 do not tolerate more than 15% ethanol by weight (96% by volume). The xanthan gum does not thicken at more than 14.4% ethanol by weight, the gels break, and the pigments do not remain in suspension.

TABLE 5

| | GO | GO | GO | GO |
|---|---|---|---|---|
| | 147\02 | 147\03 | 146\02 | 146\03 |
| Glycerin 99.5 | 5.0 | 5.0 | — | — |
| wt.-% | | | | |
| Phenoxyethanol | 0.4 | 0.4 | 0.4 | 0.4 |
| Perfume | 0.1 | 0.1 | 0.1 | 0.1 |
| Potassium | 0.1 | 0.1 | 0.1 | 0.1 |
| hydroxide | | | | |
| Ethanol 96 Vol.-% | 15.0 | 15.0 | 15.0 | 15.0 |
| denat. (t-Butanol, | | | | |
| Bitrex) | | | | |
| Dermacryl 79 | 1.0 | — | 1.0 | — |
| Colorona Precious | 3.0 | 3.0 | 3.0 | 3.0 |
| Gold | | | | |
| Tylose H 100000 | 3.0 | 3.0 | — | — |
| YP2 | | | | |
| Natrosol 250 HR | — | — | 3.0 | 3.0 |
| Isopropyl | — | 4.0 | — | 4.0 |
| myristate | | | | |
| Water, | 72.4 | 69.4 | 77.4 | 74.4 |
| demineralized | | | | |

The pigments in the compositions of table 5 were not to be kept homogeneously dispersed. The alcohol content is too low for the specified requirements for the product.

TABLE 6

| | GO | GO | GO | GO | GO |
|---|---|---|---|---|---|
| | 181\01 | 181\02 | 181\03 | 181\04 | 181\05 |
| Isopropyl myristate | 1.00 | 1.00 | 1.00 | 1.00 | 1.00 |
| Stearamidopropyl | 0.50 | 0.50 | 0.50 | 0.50 | 0.50 |
| Dimethylamine | | | | | |
| Dehyquart A CA | 1.00 | 1.00 | 1.00 | 1.00 | 1.00 |
| Perfume | 0.10 | 0.10 | 0.10 | 0.10 | 0.10 |
| Xirona Caribbean | 7.00 | 5.00 | 5.00 | 20.00 | 5.00 |
| Blue | | | | | |
| Propandiol-1.2 | 7.50 | 7.50 | 7.50 | 7.50 | 7.50 |
| Tylose H 100000 YP2 | 2.50 | 2.00 | 1.50 | 1.50 | 1.50 |
| Ethanol 96 Vol.-% | 28.40 | 30.90 | 31.40 | 31.40 | — |
| denat. (t-Butanol, | | | | | |
| Bitrex) | | | | | |
| Water, | 52.00 | 52.00 | 52.00 | 37.00 | 83.40 |
| demineralized | | | | | |

In all formulations in table 6 the pigments sank to the bottom. The gels were not stable.

TABLE 7
Raw materials used

| Trade name: | INCI | Manufacturer |
|---|---|---|
| Baycusan C 1001/1 | Polyurethane-34 (32 wt.-%), Aqua (68 wt.-%) | Covestro |
| Dermacryl 79 | Acrylates/octylacrylamide copolymer (97% | AkzoNobel |
| | by weight) | |
| Bentone 38 V CG | Disteardimonium Hectorite | Elementis |
| | | Specialties |
| Timiron | Mica (and) Titanium Dioxide (10-125 μm) | Merck |
| Snowflake MP-99 | | |
| Dehyquart A CA | Cetrimonium chloride (25% by weight), | BASF SE |
| | citric acid (1,5% by weight), aqua | |
| | (73,5% by weight) | |
| Xirona | Mica (and) Titanium Dioxide (and) Silica | Merck |
| Caribbean Blue | (and) Tin Oxide; Mica (CI 77019): | |
| | 23-42% by weight. | |
| | titanium dioxide (CI 77891): 29-39% by weight. | |
| | silica: 29-37% by weight. | |
| | tin oxide: ≤1.0 wt.-% | |
| | 10.0-60.0 μm (80% within this size range); | |
| | D50: 21.0 · 27.0 μm | |
| Xirona Golden | Silica, CI 77891 (titanium dioxide), | Merck |
| Sky | Tin Oxide (silicon dioxide coated | |
| | with titanium dioxide and tin oxide | |
| Colorona | Mica, CI 77891 (Titanium dioxide), Silica, | Merck |
| Precious Gold | CI 77491 (Iron oxides), Tin oxide | |
| Sumicos | Mica (and) Iron Oxide [10.0-60.0 μm | Sudarshan |
| Walnut 43839 | (80% within this size range) | |
| Prestige | Mica (and) Iron Oxide [10.0-50.0 μm | Sudarshan |
| Fire Red | (80% within this size range) | |
| Oxen C88046 | Mica, Calcium Aluminum | Hebei Oxen |
| Golden/Kelly/ | Borosilicate, CI 77491 (Iron oxides) | New Materials |
| Green/Blue | | |
| VOLAREST FL-LQ | Acrylates/Beheneth-25 Methacrylate | Croda |
| (RB) EN 01955 | Copolymer (30 wt.-%), Sodium Laureth | |
| | Sulfate, Aqua | |
| Keltrol CG-SFT | Xanthan gum | CP Kelco |
| Tylose H | Hydroxyethyl cellulose | ShinEtsu |
| 100000 YP2 | | |
| Natrosol 250 HR | Hydroxyethyl cellulose | Ashland |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition comprising:
from about 30 to about 85% by weight ethanol,
from about 15 to about 37% by weight water,
at least one crosslinked copolymer composed of acrylic acid and non-ethoxylated esters of acrylic acid with linear C10-C30 monoalcohols as monomers, the crosslinked copolymer being present in a total amount of from about 0.1 to about 1.0% by weight,
at least one alkalizing agent up to a pH of the cosmetic composition in the range from about 5 to about 8, preferably in the range 6-7, measured at 20° C.,
at least one substance selected from oils and fragrances in a total amount of from about 0 to about 10% by weight, and
one or more surfactants in a total quantity of from about 0 to about 0.1% by weight
at least one pigment in a total amount of from about 0.1 to about 10% by weight,
where all quantities are based on the weight of the cosmetic composition.

2. The cosmetic composition according to claim 1, wherein the alkalizing agent is selected from the group comprising alkanolamines and alkali metal hydroxides and mixtures thereof.

3. The cosmetic composition according to claim 2, wherein at least one alkanolamine chosen from 2-amino-2-methylpropan-1-ol (AMP), triethanolamine, triisopropanolamines (1,1′,1″-nitrilotris-2-propanol), tromethamines (2-amino-2-(hydroxymethyl)-1,3-propanediol), tetrahydroxypropyl ethylenediamines, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol and 2-amino-2-methylpropan-1,3-diol and mixtures thereof.

4. The cosmetic composition according to claim 2, wherein at least one alkanolamine chosen from 2-amino-2-methylpropan-1-ol, triethanolamine, triisopropanolamines (1,1′,1″-nitrilotris-2-propanol), tromethamine (2-amino-2-(hydroxymethyl)-1,3-propanediol), tetrahydroxypropyl ethylenediamine, 2-aminoethan-1-ol, and 2-amino-2-methyl-propan-1,3-diol, and mixtures thereof.

5. The cosmetic composition according to claim 1, wherein at least one substance selected from oils and fragrances is included in a total amount of from about 0.05 to about 5% by weight, based on the weight of the cosmetic composition.

6. The cosmetic composition according to claim 1, wherein at least one color, effect and/or pearlescent pigment is present as pigment, which is selected from metal oxides, metal hydroxides, metal oxide hydrates, sulfur-containing silicates, metal sulfides, complex metal cyanides, metal sulphates, bronze pigments, substrate platelets, comprising a vacuum metallized pigment (VMP) and nacreous pigments, wherein the nacreous pigments have a pigment core of a platelet-shaped material selected from natural mica, synthetic mica and borosilicates, which pigment core may be coated with one or more layers of one or more metal oxides or a metal oxychloride.

7. The cosmetic composition according to claim 1, wherein no titanium dioxide and no titanium dioxide-containing pigment is present.

8. The cosmetic composition according to claim 1, wherein the at least one pigment has an average particle size D₅₀, determined by dynamic light scattering (DLS), of from about 1.0 to about 50 μm.

9. The cosmetic composition according to claim 1, wherein the crosslinked copolymer built up from acrylic acid and non-ethoxylated esters of acrylic acid with linear C10-C30 monoalcohols as monomers is acrylates/C10-30 alkyl acrylates crosspolymer.

10. The cosmetic composition according to claim 1, wherein Polyurethane-34 is included in an amount of from about 0.03 to about to about 1% by weight.

11. The cosmetic composition according to claim 1, wherein:
the crosslinked copolymer is present in a total amount of from about 0.45 to about 0.60% by weight, and
the at least one pigment is present in a total amount of from about 3 to about 4% by weight, where all quantities are based on the weight of the cosmetic composition.
